(19) United States
(12) Patent Application Publication
(10) Pub. No.: US 2004/0044326 A1
(43) Pub. Date: Mar. 4, 2004

(54) Method for tracking bags of blood and blood products

(76) Inventors: Lewis M. Kranz, Kenosha, WI (US); Anita M. McMiller, Kenosha, WI (US); Kip Seremjian, Kenosha, WI (US); Rodney F. Alley, Kenosha, WI (US)

Correspondence Address: JANSSON, SHUPE & MUNGER, LTD, 245 MAIN STREET, RACINE, WI, 53403, US

(21) Appl. No.: 10/232,294

(22) Filed: Aug. 30, 2002

(51) Int. Cl. A61B 19/00

(52) U.S. Cl. 604/408

(57) ABSTRACT

The invention involves a method for tracking bags containing biological fluids for dispensing within a human body, including, but not limited to human blood and plasma. The method comprises the steps of: obtaining a bag configured and arranged for sterile, aseptic, and stable retention of biological fluids; placing a quantity of the biological fluids into the bag; providing a first information-processing device with a data-input device and with a data-transfer device, said data-transfer device capable of writing data that has been input into the information-processing device, into a data-storage chip in a manner readable by a second information-processing device; affixing to the bag a readable/writeable data-storage chip; collecting fluid-data about the biological fluid; and inputting data, such as the fluid-data, into the first information-processing device for data transfer onto the chip. The readable/writeable data-storage chip is capable of interfacing with the first and second information-processing devices. The first information-processing device can write data to the data-storage chip and the second information-processing device can read of the data on the chip.

### FIELD OF THE INVENTION

This invention relates to storage containers for blood and blood products, and more specifically to the tracking of storage containers containing blood and blood products.

### BACKGROUND OF THE INVENTION

Each year within the United States, millions of units of whole blood and blood products are transfused into donees. Dispute its near ubiquity and seeming ease of harvesting, blood is a highly-regulated product and carries with it bio-hazard considerations.

Due to the potentially devastating effects of introducing donor blood into a person through a transfusion, thereby bypassing the body's primary means of defense, blood products are highly regulated. Such concerns include compatibility, freedom from contagion, and viability.

It has long been known that transfusion of incompatible blood intended to be lifesaving can be life-threatening. Compatibility (through typing and cross-matching) must be determined prior to transfusion. Typing is the in vitro testing performed on a donor's blood with respect to determine reaction of the blood to known reagents. Cross-matching is the observation of in vitro mixing of donor's and donee's blood in a manner for determination of agglutination, coding, and hemolytic antibodies. Once compatibility information is determined, that information must be recorded.

Typing and cross-matching includes determination of gross factors such as blood type (O, A, B, and AB), the Rh factor (positive or negative), and compatibility with individual characteristics of particular blood, such as antibodies, which can establish subtypes. Inherited and acquired characteristics can result in a rare blood type is any type that is hard to find. A blood type is considered rare when more than two hunderd donors have to be screened to find one compatible donor.

Separate from the compatibility question is the question of freedom from contagion. History is replete with the introduction of serious, chronic, and life-threatening disease introduced into a patient through transfusion of tainted blood or blood products. Blood-borne diseases are legion and include Human Immunodeficiency Virus (HIV) and Hepatitis types A, B, and C.

As an organic product, blood and blood products are subject to decomposition over time. To prolong viability of a sample harvested from an otherwise healthy human, preservatives are typically enclosed in collection kits. It is well known that the storage of certain blood components in plastic bags can be enhanced by providing certain chemicals in the storage solution. For example, the control of pH of platelet concentrates is critical to long term storage and the pH can be controlled with certain buffers such as bicarbonate salts in solution. Ideally, such compounds should be available in a “closed” blood bag storage system.

Preservatives, however, will not protect a sample for prolonged time nor to changes in environmental conditions, such as temperature, which might not otherwise destroy the container holding the blood but can damage the blood within. It is thus extremely important once blood is harvested into a storage unit, to clearly mark the unit with compatibility markers, the harvest date (or in the alternative, the expiration date), and a certification as to the cotangent testing (or in the alternative the donor from whom the sample was obtained).

After harvesting, the blood or blood product is stored in a container. It is necessary to mark the container with such identifying information. Once the blood is placed in the bag, it is important to make certain that the identifying information is not changed, either inadvertently or intentionally, in such a manner as to make the blood or blood product deadly to a potential donee.

Moreover still, using typical collection bags, receipt of a bag at a given temperature does not guarantee that the sample was maintained at an advantageous temperature throughout its storage life. A visual inspection of the bag may reveal blood which appears whole and healthy but may have started to decompose due to high-temperature storage.

Even given a container of blood or blood products which is properly marked and properly stored, unfortunately it is not uncommon for the blood or blood products to be negligently transfused into an unsuspecting donee.

Separate from the issue of compatibility of a pre-harvested sample into a donee patient in a clinical setting by a healthcare provider, there are opportunities for direct, interpersonal, transfusion. Such circumstances could occur, for example, in a field situation for military personnel. Currently, such gross typing information is recorded on identification tags (known as dog tags) for military personnel. Such gross information is valuable to untrained personnel for transfusion from a donor of one blood type to a donor of the same blood type. Such dog tags do not assist untrained personnel in interpersonal transfusion which is otherwise available in acceptable cross-type transfusions. For example, persons of A, B or AB gross blood types may receive O type blood. Conversely, persons of AB under exceptional circumstances may accept blood from O, A, or B type donors without ill effect.

### OBJECTS OF THE INVENTION

It is an object of the invention to provide an improved blood-tracking system overcoming some of the problems and shortcomings of the prior art, including those referred to above.

Another object of the invention is to provide a tamper-evident process for the transmission of blood and other biological fluids.

Another object of the invention is to provide a method of monitoring critical environmental factors of blood and other biological fluids in the process of storage and transportation.

Still another object of the invention is to provide a method to quickly determine the health safety of the donee of blood and other biological fluids given a pre-packaged quantity of blood and other biological fluids with pre-determined compatibility characteristics.

Yet another object of the invention is to provide a method to quickly determine the health safety to the donee with pre-determined compatibility characteristics of blood and other biological fluids from an interpersonal donation from a donor person with pre-determined compatibility characteristics.

Another object of the invention is to provide a method to alert health care providers to dangerous incompatibility to a donee from pre-packaged blood and other biological fluids of known characteristics.

How these and other objects are accomplished will become apparent from the following descriptions and the drawings.

### SUMMARY OF THE INVENTION

The invention involves a method for tracking bags containing biological fluids for dispensing within a human body including, but not limited to, human blood and plasma. The method comprises the steps of: obtaining a bag configured and arranged for sterile, aseptic, and stable retention of biological fluids; placing a quantity of the biological fluids into the bag; providing a first information-processing device with a data-input device and with a data-transfer device, said data-transfer device capable of writing data that has been input into the information-processing device, into a data-storage chip in a manner readable by a second information-processing device; affixing to the bag a readable/writeable data-storage chip; collecting fluid-data about the biological fluid; and inputting data, such as the fluid-data, into the first information-processing device for data transfer onto the chip. The readable/writeable data-storage chip is capable of interfacing with the first and second information-processing devices. The first information-processing device can write data to the data-storage chip and the second information-processing device can read the data on the chip.

It is preferable that the data-storage chip is of the type further having a microprocessor, an environmental-factor detector for detecting environmental factors, and an environmental-factor recorder capable of recording of the environmental factors in a form readable by the second information-processing device. In this way, environmental factors may be systematically recorded on the data-storage chip. It is preferable that the method further comprises the step of programming the second information-processing device to compare fluid-data with fluid-specific storage criteria for determining health-threatening bag-storage conditions. It is yet more preferable for the second information-processing device to have an alerting member for alerting an operator of health-threatening incompatibility.

In another embodiment, the data-storage chip is integral with the bag.

In yet another embodiment, the fluid-data includes blood-typing characteristics of blood cells contained within the bag. It is further preferred that the data-storage chip has a lockout feature prohibiting the writing over fluid-data already written onto the data-storage chip.

It is yet more preferred to provide a patient-information chip containing patient-specific information in a read-only format for reading by the second information-processing device, and to then program the second information-processing device to compare fluid-data with patient-specific information for health-threatening incompatibility. This chip would be mounted to a person. This mounting could be in the form, for example, of a dog-tag-like necklace, a wrist bracelet, or sub-cutaneous installation. The second information-processing device could have an alerting member, such as a bell, digital display, electronic lock-out, etc., for alerting an operator of health-threatening incompatibility.

In another version of this embodiment, the fluid-data includes blood-transfusion-compatibility characteristics of the blood cells. It is more preferable in this version to further provide a patient-information chip for placement about a person. The chip would contain patient-specific information in a read-only format for reading by the second information-processing device. The second information-processing device is then programmed to compare fluid-data with patient-specific information for health-threatening incompatibility.

Another aspect of this invention is a method for electronic determination of compatibility of blood for urgent direct interpersonal transfusion. Blood characteristics are pre-determined for each of two individuals. First, the blood compatibility characteristics of the blood of the two persons are determined. These blood compatibility characteristics are inscribed of the first person on data-receiving devices in a manner capable of retrieval by a data-processing device. The first data-receiving device with the blood characteristics of the first person is affixed with respect to the first person; the second data-receiving device with the blood characteristics of the second person is affixed with respect to the second person. The data-processing device is configured to compare the blood compatibility characteristics inscribed on the first and second data-receiving devices for health-threatening incompatibility. The blood compatibility characteristics of the first person and the second person input into the data-processing device. In this way, the blood compatibility characteristics of the first and second persons may be compared for health-threatening incompatibility.

It is preferred for the data-processing device to have an alerting member for alerting an operator of health-threatening incompatibility. It is more preferred for the the first and second data-receiving devices to be computer chips. These computer chips could be computer chips attached to metallic military identification tags worn by respective personnel.

It is also preferred for the first and second data-processing device each to be microcans.

Yet another aspect of the invention is a method for tracking the blood of an identified blood donor. This aspect has the steps of placing the donor's blood into a sterile blood-storage bag; securely attaching a computer chip with respect to the bag; and writing a unique donor-identifying code onto the chip in a manner readable by a data-retrieving device.

### BRIEF DESCRIPTION OF THE DRAWINGS

FIG. 1 is a perspective view of a blood bag of the current art.

FIG. 2A is a front view of a preferred embodiment of this invention.

FIG. 2B is an exploded front view of a preferred embodiment of this invention.

FIG. 2C is an exploded front view of another preferred embodiment of this invention.

FIG. 3 is a front view of another preferred embodiment of this invention.

FIG. 4A is an exploded view of a first embodiment the tab of the bag of FIG. 3.

FIG. 4B is an exploded view of a second embodiment the tab of the bag of FIG. 3.

FIG. 5 is a front view of a microcan of FIG. 4.

FIG. 6 is a perspective view of the microcan reader.

FIG. 7 is a schematic view of the method of tracking.

FIG. 8 is a front view of a microcan attached to a military identification tag.

FIG. 9 is a side view of a microcan attached to a patient's bracelet.

### DETAILED DESCRIPTION OF PREFERRED EMBODIMENTS

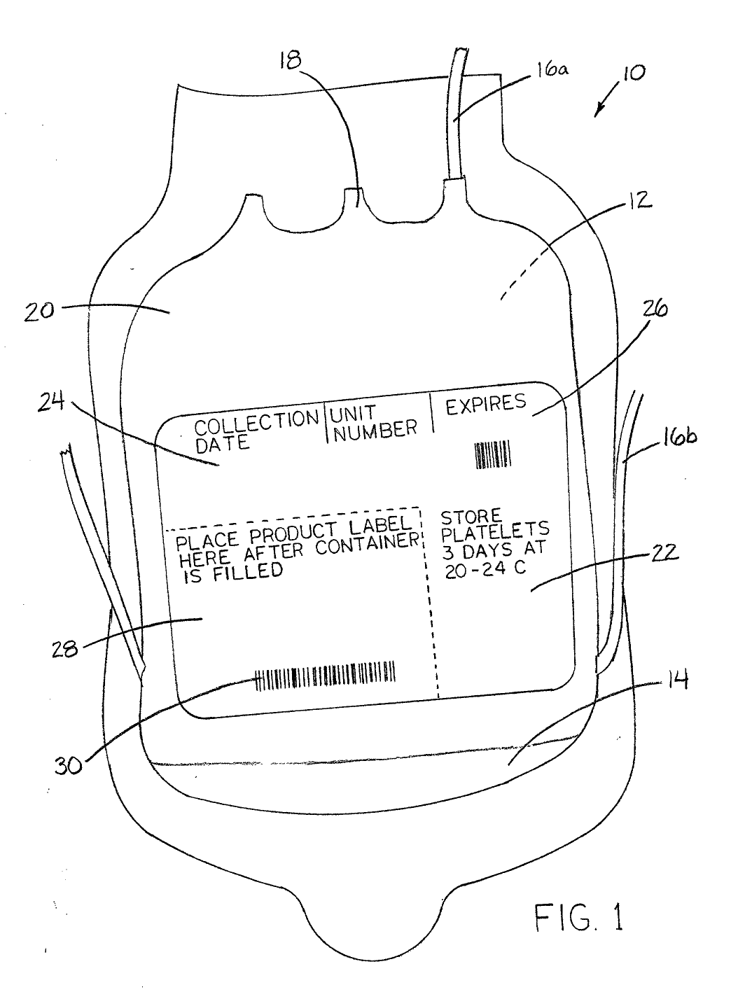

FIG. 1 shows a packed-blood-cell-delivery bag 10 of the current art. Bag 10 is typically made of a medical-grade PVC, designed to be flexible and collapsible. Bag 10 has a sterile interior 12 for receipt of blood which has been harvested from a donor. Pre-placed within interior 12 are stabilizers 14. Stabilizers 14 include anticoagulants and/or preservatives. Examples of stabilizers 14 currently in use include sodium citrate, dextrose, citric acid, monobasic sodium phosphate, and adenine.

Extending from bag 10 and in communication with interior 12 are tubes 16. Inlet tube 16a allows introduction of a donor's blood into interior 12. Outlet tube 16b allows for decanting of blood fluids after separation of whole blood into component products by centrifuging, if desired. Blood is dispensed from interior 12 of bag 10 through dispensing port 18. Other tube options are available in such bags 10, the description of which are not necessary for further discussion of the method.

Attached to exterior 20 of bag 10 is at least one label 22. Label 22 is typically designed to be moisture resistant and non-peelable. Label 22 has collection-date-recording area 24, expiration-date-recording area 26. Label 22 also has at least one blood-product-denomination-sticker-receiving area 28 for receipt of a product sticker for fixation with adhesive to label 22. Label 22 may also have pre-printed bar codes 30, uniform in coding for a bag-production run, to identify the lot of manufacture of the bag 10, or the common source of the blood-containing bags 10.

The current method of labeling and tracking consists of hand inscribing the date of collection in collection-date-recording area 24. The expiration date is calculated based on the collection date and hand inscribing said expiration date in the expiration-date-recording area 26. The blood type is determined. Blood product is determined. A blood-product-denomination-sticker is computer-generated for affixing to the bag 10. Blood-product-denomination-sticker may have a computer-generated bar code to assist in tracking inventory in a particular facility; such tracking, when it occurs, is generally isolated within a particular facility.

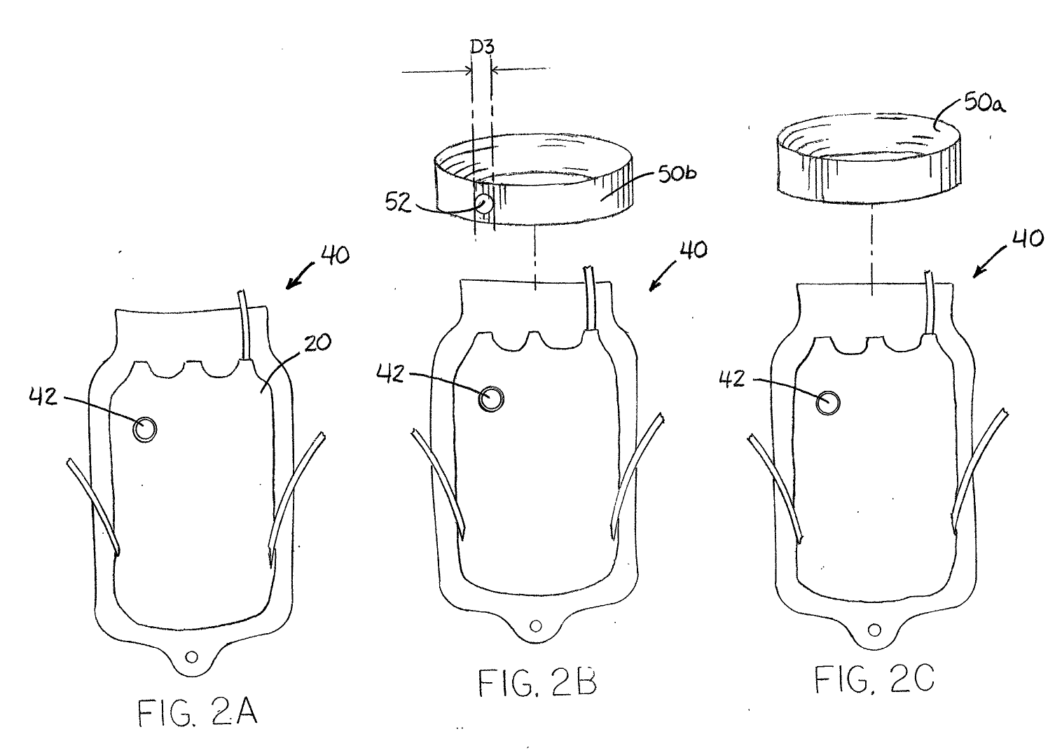

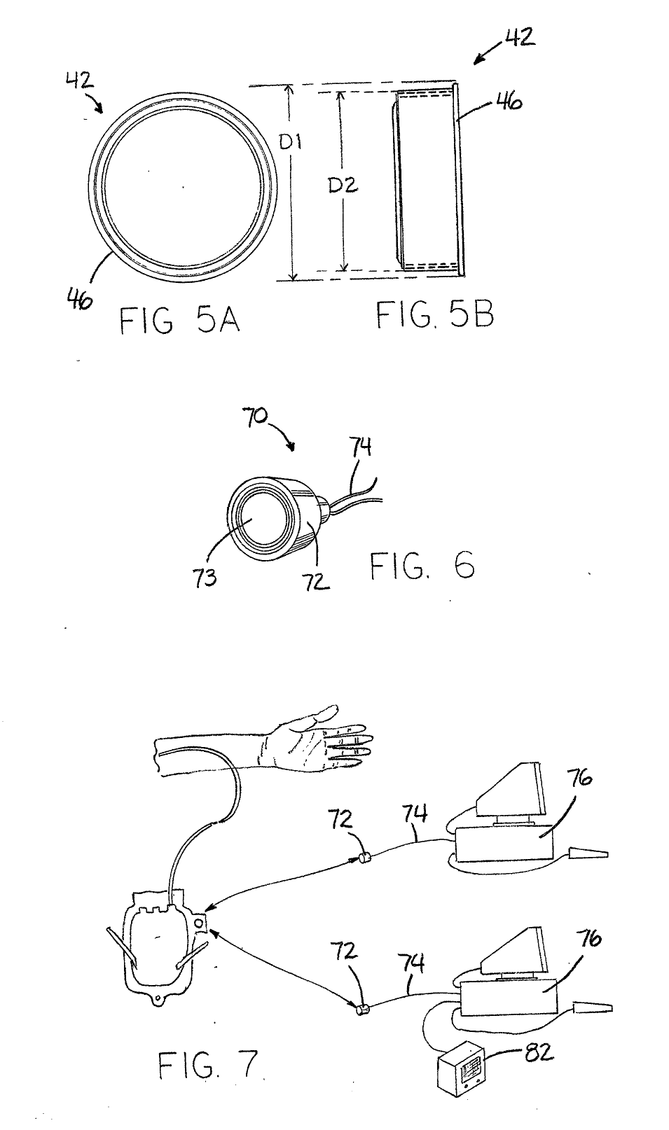

FIG. 2A is a front view of a chip-retaining bag 40 which is a preferred embodiment for use with this invention. A microcan 42 is attached by means of adhesive to the exterior 20 of the chip-retaining bag 40. A read/write microcan 42 is a non-volatile-memory computer-chip-containing button having a casing 44 of stainless steel for positive electrical conductivity properties. For some applications, it may be powered by a lithium battery. As better seen in FIGS. 5A and 5B, microcan 42 is generally cylindrical having a main-body diameter D1. Such microcans 42 have a flange 46 at the back cylindrical perimeter with a flange-diameter D2. Microcans 42 have a low profile measured in the axial direction. A suitable microcan 42, such as the Touch Memory F5 model of Dallas Semiconductors, has main-body diameter D1 of 16.25 millimeters and flange-diameter D2 of 17.35; it has an overall axial length of 5.89 millimeters, with a flange-height of 0.50 millimeters.

While it is possible to attach microcan 42 to the exterior 20 of chip-retaining bag 40 with adhesive or a pop-rivet, it is more preferable to attach microcan 42 in a tamper evident manner. One means of tamper evident attachment is through a sleeve as shown in FIGS. 2B and 2C. Thermoplastic sleeve 50a can entirely, snugly encase microcan 42 and gird it to chip-retaining bag 40. Alternatively, sleeve 50b can be of a PVC of the same type as the chip-retaining bag 40, having a sleeve aperture 52 with sleeve-diameter D3 greater than that of the casing main-body diameter D1 to accommodate passage of the microcan 42 body, but less than the flange diameter D2 to retain the flange 46. The sleeve 50b is then heat sealed to the chip-retaining bag 40.

In these manners, the removal and replacement of microcan 42 will be evident by visual inspection as it will be necessary to damage sleeve 50a or 50b in order to separate microcan 42 from chip-retaining bag 40.

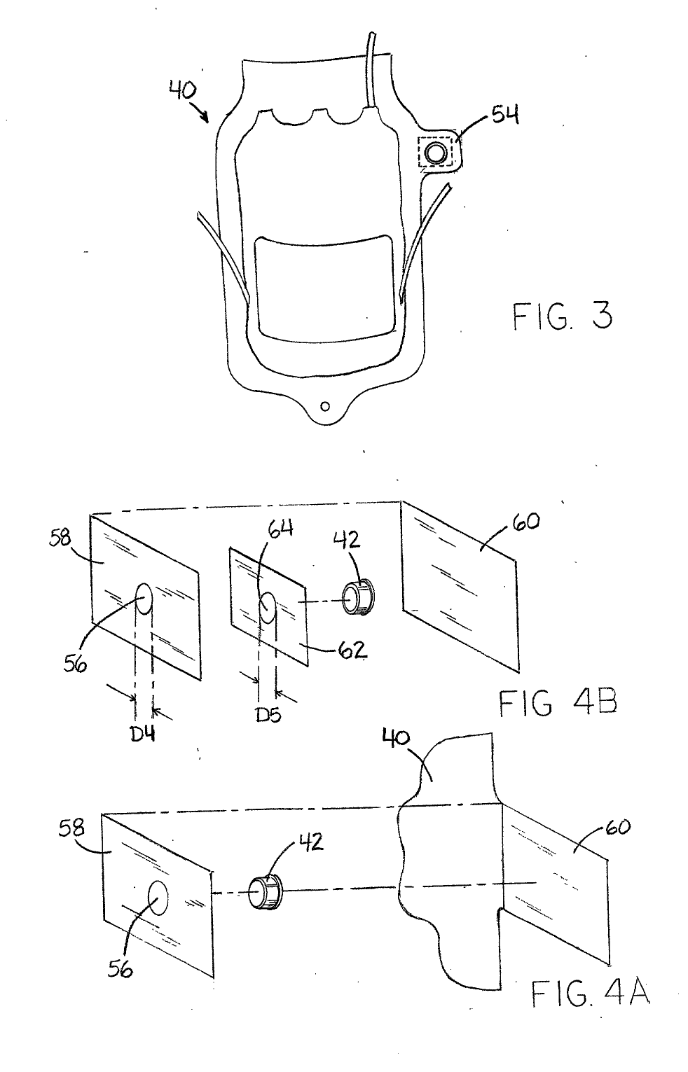

FIG. 3 shows another preferred embodiment of this invention. As shown in FIG. 4A, chip-retaining bag 40 includes an integral tab 54. By the term “integral”, inventors mean that tab 54 may either be continuous with bag 40, a portion of tab 54 may be continuous with bag 40, or tab 54 may be a separate piece subsequently attached to bag 40. Tab 54 may be created by two pieces of the same PVC material of chip-retaining bag 40, creating a tab front 58 and a tab back 60 of the same planar dimensions. Tab front 58 has a tab aperture 56 of a diameter D4 which is greater than that of main-body diameter D1 of microcan 42, but less than diameter D2 of outer flange 46, to retain microcan 42 within tab 54. Before integration with chip-retaining bag 40, microcan 42 is slid through tab aperture 56 such that only flange 46 is retained between tab front 58 and tab back 60. By the phrase “integration with chip-retaining bag 40”, inventors mean sealing the edges of tab front 58 to tab back 60 to create a pocket while previously, simultaneously, or subsequently fusing tab 54 (or a portion thereof, as appropriate) to chip-retaining bag 40.

FIG. 4B shows an alternate means of securing microcan 42 to bag 40 with tamper-evident security. As seen in FIG. 4B, an apertured metal yoke 62 is fashioned from a thin plate to be smaller in each planer dimension than tab 54. In one version, apertured metal yoke 62 has yoke aperture 64 of a diameter D5 which is greater than that of main-body outer diameter D1 of microcan 42, but less than flange diameter D2, to retain microcan 42 within tab 54. As shown in FIG. 4B, microcan 42 is slid through yoke aperture 64 and then tab aperture 56 of tab front 58. In the same manner as discussed above, tab front 58 is sealed to tab back 60 and integrated with chip-retaining bag 40, but in such a manner that yoke is entirely retained within tab 54 and flange 46 is retained between metal yoke 62 and tab back 60. (Apertured yoke 64 is shown in phantom in tab 54 of FIG. 3.)

Other secure methods of affixing microcan 42 to bag 40 may be used without going out of the scope of the present invention.

FIGS. 5A, 5B and 6 show microcan 42 and a microcan reader 70, respectively. Microcan 42 has 4K-bit read/write memory plus a unique 48-bit serial number in read-only format. For some applications described herein, microcan 42 may also contain a lithium battery and environmental sensors. Environmental factors which can be monitored include: temperature, pressure, altitude, and conductivity. Microcans 42 may be mechanically locked, if desired in certain applications consistent with the methods of this invention, to prevent over-writing.

Microcan readers 70 are currently known in the industry, such as those made by: Psion, PLC; Telxon; Symbol Technologies, Inc.; and Videx. An example of a suitable reader 70 is the hand-held, computer peripheral iButton Probe. These standard microcan readers 70 typically have an interface portion 72 consisting of a concavity 73 complementary to the casing 44 of microcan 42. Interface portion 72 is either integral with or attached by means of a transmission cable 74 (or radio wave carrier) to a data-receiving/generating portion 76 (shown in FIG. 7). Data-receiving/generating portion 76 may be, by way of example only, a personal computer or a handheld computer.

FIG. 7 shows a schematic of the method of this invention. The microchip contained in microcan 42 is encoded with certain information such as the bag-manufacturer's lot number and stabilizer lot number. Data-receiving/generating portion 76 is regularly up-dated with rejection information such as manufacturer's recall of a production lot of chip-retaining bags 40. Prior to donation of blood, microcan reader 70 reads microcan 42 on chip-retaining bag 40 to verify that bag 40 has not been recalled for any reason.

Prior to donation, the donor is screened. By way of example, under current Department of Defense donor deferral rules, all potential donors will be deferred indefinitely due to variant Creutzfeldt-Jakob Disease (the human form of “mad cow” disease) if they have: (1) traveled or resided in the United Kingdom for a cumulative total of three months or more at any time from 1980 through the end of 1996; (2) received a blood transfusion in the U.K. at any time from 1980 to the time of donation; (3) traveled to or resided anywhere in Europe for a cumulative total of six months or more at any time from 1980 through the end of 1996; or (4) traveled to or resided anywhere in Europe for a cumulative total of five years or more at any time from Jan. 1, 1997, to the time of donation. History of other disease such as Babesiosis (which is a parasitic infection) will result in a permanent deferral.

Blood is then introduced into interior 12 of bag 40 from a donor through inlet tube 16a. Prior to or contemporaneous with donation of the blood, the blood type of the donor is determined and microcan 42 is inscribed with optional data by microcan reader 70. Optional data fields may include the date of blood collection, blood type of donor, anonymous blood-donor identification number (in allogeneic donations which refer to blood tranfused into someone other than the donor; such number would be used on all blood donated by this donor after initial assignment of the number), institution of collection, and place of donation. Further fields could include the name of the donor (especially in cases of autologous transfusions which refers to donations where the blood donor and the donee are the same). An expiration date can either be input as a data-entry field or calculated automatically by the computer based on the date of collection.

Blood is also screened for infectious disease. Currently, nine tests for infectious disease are conducted on each unit of donated blood: Hepatitis B surface antigen, antibodies to Hepatitis B core, antibodies to Hepatitis C virus, antibodies to the human immunodifficiciency virus, types 1 and 2, HIV-1 (which tests for antigens of HIV-1 virus), antibodies to human T-lymphotrophic virus (types 1 and II), syphilis, and nucleic acid amplification.

Rather than assign a number for the allogeneic donor for permanent future use on all blood harvested from that donor, as microcan 42 comes preloaded with a read only, unique serial number, it is possible to assign the particular serial number to an individual donor on records kept at the place of collection. In this way, the donor will remain anonymous to any person receiving the blood-containing chip-retaining bag 40 as it is stored and transported yet, should the donor be subsequently diagnosed with a blood-borne contagion, the particular preloaded serial number can be added to a blood-donor-reject database such that chip-retaining bag 40 can be discarded prior to infiltration into a donee.

Regardless of whether donor has a permanent identification or is identified by microcan serial number, if the donor is permanently or temporarily deferred, then the donor's identifying information will be transmitted to all data-receiving/generating portions 76.

Once the blood product has been collected and data inscribed on microcan 42, the blood within chip-retaining bag 40 may be manipulated to create various blood products. Because patients seldom require all of the components of whole blood, it makes sense to transfuse only that portion needed by the patient for a specific condition or disease. This treatment, referred to as “blood component therapy”, allows several patients to benefit from one unit of donated whole blood. Blood components include red blood cells, plasma, platelets, and cryoprecipated antihemophilic factor. Up to four components may be derived from any one unit of blood. Microcan 42 will withstand pressures to which chip-retaining bag 40 is subjected in a centrifuging process commonly used to separate components. Plasma may be decanted off into another blood product chip-retaining bag 40. At that time, microcan 42 of the original chip-retaining bag 40 may be read by microcan reader 70 and the information inscribed onto another, or “daughter” microcan 42 on a second chip-retaining bag 40a by the same microcan reader 70. The information copied onto daughter microcan 42 could include the original patient number from first microcan 42. In this way, all product obtained from one contaminated source can ultimately be tracked and isolated. Moreover, any adverse environmental conditions to which the mother chip-retaining bag 40 was subjected would be recorded onto the daughter microcan 42 of the daughter chip-retaining bag 40a.

Blood-containing chip-retaining bag 40 is transported to a health-care-providing location for transfusion into a donee.

Prior to cross-matching, microcan 42 on chip-retaining bag 40 is read by microcan reader 70 (most typically of a hand-held variety for convenience) at a health-care-providing location. If information contained on microcan 42 is in the rejection database, due either to the recall of chip-retaining bag 40 or the deferral of the donor, the workers at the health-care facility are alerted. The alert can be delivered through an alarm 82 such as an audio speaker attached to a personal computer, shown in FIG. 7.

Moreover, various embodiments of microcans 42 currently available have as an option an environmental monitor to discover and record environmental conditions, such as temperature, throughout storage and transportation of the blood-containing chip-retaining bag 40. Of those microcans 42 having environmental control detectors, storage conditions would be recorded on the microchip within microcan 42, as well. If environmental health-threatening factors are recorded, the heath-care workers would be alerted.

The memory capacity of microcan 42 allows for encoding of an optional patient-verification-code at the laboratory after cross-matching a sample of blood from the blood-containing chip-retaining bag 40 with donee's blood.

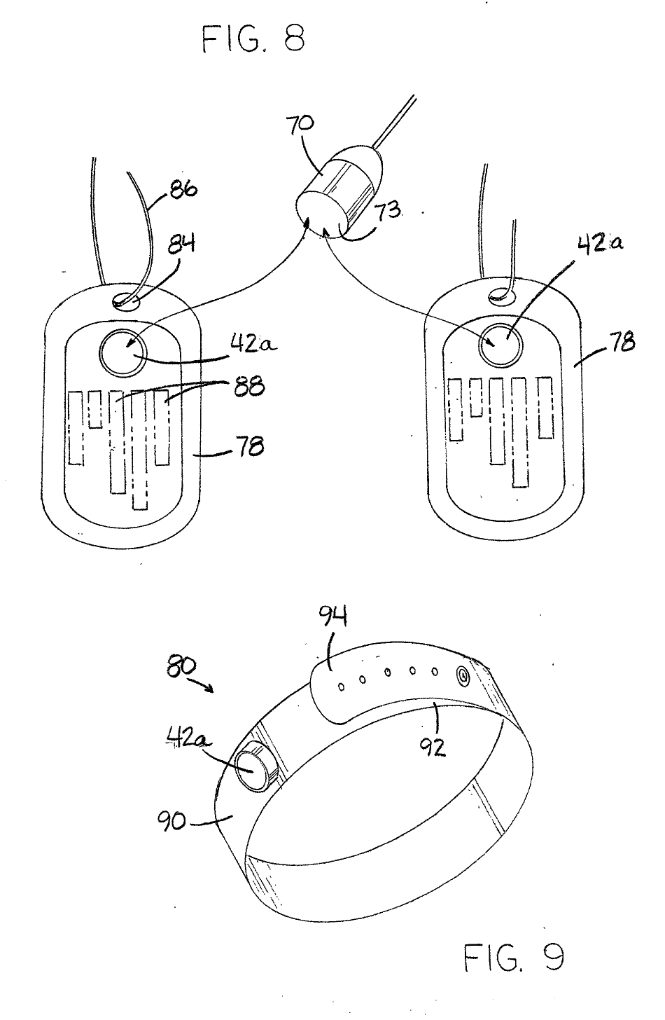

Preassigned to donee and attached to donee at health-care-providing location is a donee microcan 42a. Donee microcan 42a is the same type as on chip-retaining bag 40, but affixed to soldier's military identification tag 78 (as shown in FIG. 8) or a patient's hospital bracelet 80 (as shown in FIG. 9).

Prior to transfusion into donee, microcan reader 70 reads both microcans 42, 42a from blood-containing chip-retaining bag 40, and from patient (mounted on either identification tag 78 or bracelet 80), and compares gross blood type and verification code if provided. Health-care workers would be alerted by alarm 82 if the blood was incompatible or directed to the wrong patient.

FIG. 8 illustrates another method of this invention to assure relative safety of in-the-field person-to-person transfusions. Prior to assignment into the field, each participant will be assigned a personal identification token such as metal identification tags 78. Tag 78 has a chain aperture 84 to accommodate a chain 86 for wearing around a participant's neck. Tag 78 has identifying-information areas 88 for visual identification information such as name, position, and identifying number of the participant. Microcan 42 is attached to tag 78 by any suitable means. Microcan 42 is encoded with the participant's name, gross blood type, and any donor-deferral information.

In in-field emergency medical situations where a person-to-person transfusion is necessary, a health-care provider can simply touch a microcan reader 70 to the potential donor's and donee's microcans 42a on tags 78 in seriatim, allowing reader 70 to instantly determine compatibility.

FIG. 9 shows yet another vehicle for providing personal identification utilizing an identification band, such as those bracelets commonly used in hospitals. Bracelet 80 is made of flexible plastic strip 90 having a first end 92 and a second end 94. Between first end 92 and second end 94 is an attached microcan 42a containing identifying information including participant's name and blood type. Microcan 42a is positioned such that it faces in an outward manner and may be read by a microcan reader 70. Strip ends 92, 94 are secured by a locking member which clamps strip end 94 to strip end 92 at an appropriate circumference to avoid falling off. Such strips 90 may be secured to any appropriate portion of the body such as wrist or ankle.

While the principles of the invention have been shown and described in connection with specific embodiments, it is to be understood that such embodiments are by way of example and are not limiting.

We claim:

1. A method for tracking bags containing biological fluids comprising the steps of:
obtaining a bag configured and arranged for sterile retention of fungible biological fluids for dispensing within a human body;
placing a quantity of fungible biological fluids for dispensing within a human body, into the bag;
providing a first information-processing device with a data-input device and with a data-transfer device, said data-transfer device capable of writing data that has been input into the information-processing device, into a data-storage chip in a manner readable by a second information-processing device;
affixing to the bag a readable/writeable data-storage chip capable of interfacing with the first and second information-processing devices for writing of data from the first information-processing device to the readable/writeable data-storage chip and for reading of the data by the second information-processing device;
collecting fluid-data about the biological fluid; and
inputting the fluid-data into the first information-processing device for writing of the fluid-data onto the chip.

2. The method of claim 1 wherein the data-storage chip is of the type further having a microprocessor, an environmental-factor detector for detecting environmental factors, and an environmental-factor recorder for recordation of the environmental factors in a form readable by the second information-processing device, further comprising the step of systematically recording environmental factors on the data-storage chip.

3. The method of claim 2 further comprising the steps of:
programming the second information-processing device to compare fluid-data with fluid-specific storage criteria for determining health-threatening bag-storage conditions.

4. The method of claim 3 wherein the second information-processing device has an alerting member for alerting an operator of health-threatening incompatibility.

5. The method of claim 1 wherein the data-storage chip is integral with the bag.

6. The method of claim 1 wherein the fluid contains blood cells and the fluid-data includes a blood type of blood cells contained within the bag.

7. The method of claim 6 wherein the data-storage chip has a lockout feature prohibiting the writing over fluid-data already written onto the data-storage chip.

8. The method of claim 7 further comprising the steps of:
providing a patient-information chip mounted with respect to a person, containing patient-specific information in a read-only format for reading by the second information-processing device;
programming the second information-processing device to compare fluid-data with patient-specific information for health-threatening incompatibility.

9. The method of claim 8 wherein the second information-processing device has an alerting member for alerting an operator of health-threatening incompatibility.

10. The method of claim 7 wherein the fluid-data includes blood-transfusion-compatibility characteristics of the blood cells.

11. The method of claim 10 further comprising the steps of:
providing a patient-information chip mounted with respect to a person, containing patient-specific information in a read-only format for reading by the second information-processing device;
programming the second information-processing device to compare fluid-data with patient-specific information for health-threatening incompatibility.

12. A method for determination of compatibility of blood of pre-determined characteristics of a first human and of blood of pre-determined characteristics of a second human for urgent direct interpersonal transfusion comprising the steps of:
determining blood compatibility characteristics of the blood of the first person and of the second person;
inscribing the blood compatibility characteristics of the first person on a first data-receiving device and the blood compatibility characteristics of the second person on a second data-receiving device, in a manner capable of retrieval by a data-processing device;
affixing the first data-receiving device with respect to the first person and the second data-receiving device with respect to the second person;
programming the data-processing device to compare the blood compatibility characteristics inscribed on the first and second data-receiving devices for health-threatening incompatibility; and
retrieving the blood compatibility characteristics of the first person and the second person into the data-processing device;
whereby the blood compatibility characteristics of the first and second persons may be compared for health-threatening incompatibility.

13. The method of claim 12 wherein the data-processing device has an alerting member for alerting an operator of health-threatening incompatibility.

14. The method of claim 13 wherein the first and second data-receiving devices are first and second computer chips, respectively.

15. The method of claim 14 wherein the blood-characteristic-inscribed first and second computer chips are attached with respect to the first and second persons by first and second personal identification tags.

16. The method of claim 12 wherein the first and second data-processing device are each microcans.

17. A method for tracking the blood of an identified blood donor, comprising the steps of:
placing blood from the identified blood donor into a bag configured for receipt and sterile storage of blood;
undetachably attaching a computer-readable/writeable data chip with respect to the bag;
assigning a unique donor-identifying code to the donor; and
writing the donor-identifying code onto the chip in a manner readable by a data-retrieving device,
whereby the blood within the bag can be distinguished.
